(19) United States
(12) Patent Application Publication
(10) Pub. No.: US 2002/0087079 A1
(43) Pub. Date: Jul. 4, 2002

(54) Position sensitive catheter having scintillation detectors

(76) Inventors: Leon Kaufman, San Francisco, CA (US); Vartan Ghazarossian, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/754,074

(22) Filed: Jan. 3, 2001

(51) Int. Cl. A61B 6/00

(52) U.S. Cl. 600/436

(57) ABSTRACT

The present invention relates generally to in vivo evaluation of labeled lesions within a body lumen using a catheter having an array of radiation detectors. The present invention provides catheters and methods which detect radiotracers which have bound to vulnerable plaque. The radiation detectors can be coupled to a signal processor through a delay line so as to reduce the number of transmission lines traveling through the catheter to the outside of the body. The radiation detectors can be semiconductor detectors or optical detectors.

### CROSS REFERENCE TO RELATED APPLICATIONS

The present application is related to Patent Application No.___________ , filed herewith, entitled “Position Sensitive Catheter” (Attorney Docket No. 020039-000610US), the complete disclosure of which is incorporated herein by reference.

### BACKGROUND OF THE INVENTION

The present invention relates generally to medical devices. More particularly, the present invention relates to methods, systems, and kits for detecting radiotracers attached to atherosclerotic plaque within a body lumen.

Coronary artery disease resulting from the build-up of atherosclerotic plaque and its subsequent rupture to form blood-flow blocking clots in the coronary arteries is the leading cause of death in the United States. The plaque build-up leads to a narrowing of the artery and as a consequence, reduces the blood flow to the myocardium (i.e., heart muscle tissue). Myocardial infarction, better known as a heart attack, can occur when the arterial plaque abruptly closes a vessel, causing complete cessation of blood flow to portions of the myocardium, or more likely the plaque ruptures, fissures, or erodes and produces a clot that blocks the vessel at that point or distally (downstream). Even if abrupt closure does not occur, blood flow may decrease resulting in chronically insufficient blood flow which can cause significant tissue damage over time.

A variety of interventions have been proposed to treat coronary artery disease. For disseminated disease, the most effective treatment is usually coronary artery bypass grafting (CABG) where problematic lesions in the coronary arteries are bypassed using external grafts. In cases of less severe disease, pharmaceutical treatment is often sufficient. Finally, focused disease can often be treated intravascularly using a variety of catheter-based approaches, such as balloon angioplasty, atherectomy, radiation treatment, stenting, and sometimes combinations of these approaches.

With a variety of treatment techniques which are available, the physician is faced with a challenge of selecting the particular treatment which is best suited for an individual patient. While numerous diagnostic aids have been developed, no single technique provides all of the information which is needed to select the optimum treatment. Angiography is very effective in locating the lesions in the coronary vasculature, only of these plaques intrude into the lumen, furthermore provides little information concerning the characteristics of the plaque. To provide better characterization of the lesion(s), a variety of imaging techniques have been developed for providing a more detailed view of the lesion, including intravascular ultrasound (IVUS), angioscopy, laser spectroscopy, computer tomography (CT), magnetic resonance imaging (MRI), or the like. None of these techniques, however, are completely successful in determining the exact nature of the lesion. In particular, such techniques provide little information regarding whether the plaque is stable or vulnerable.

Depending on the type of plaque present in the coronary arteries and other vessels, it may contain, among other components: inflammatory cells, smooth muscle cells, cholesterol, and/or fatty substances. These materials are usually trapped between the endothelium of the blood vessel and the underlying smooth muscle cells. Depending on various factors, including thickness, composition, and size of the deposited materials, the plaques can be characterized as stable or vulnerable. The plaque is normally covered by an end cap. However, under certain adverse conditions the cap can be disrupted, leading to the release of thrombogenic material which is capable of activating the clotting cascade and inducing coronary thrombosis. Such plaque is referred to as vulnerable plaque. The resulting thrombus caused by the vulnerable plaque can cause angina chest pain, acute myocardial infarction, stroke, or sudden coronary death. It has recently been proposed that plaque instability, rather than the degree of plaque build-up, should be the primary determining factor for treatment selection.

A variety of approaches for detecting vulnerable plaque in patients have been proposed. One technique involves detecting a slightly elevated temperature within vulnerable plaque resulting from the inflammation. Another technique involves interrogation of the plaque by infrared light. It has also been proposed to introduce radiolabeled material which has been shown by autoradiography to bind to stable and vulnerable plaque in different ways. External detection of the radiolabels, however, greatly limits the sensitivity of these techniques and makes it difficult to determine the precise locations of the affected regions. Thus far, none of these techniques have possessed sufficient sensitivity or resolution necessary to reliably characterize the vulnerable plaque at the cellular level in the blood vessel.

For all of the above reasons, what is needed are methods and systems which accurately differentiate and measure the vulnerable plaque in vivo.

### SUMMARY OF THE INVENTION

The catheters of the present invention provide for in vivo detection of radio labels disposed within a body lumen. In one aspect, the present invention provides catheters which include an array of radiation detectors that convert the radiation into electrical signals within the catheter and deliver the electrical signal to a signal processor that is outside of the catheter. In another aspect, the catheters of the present invention will have an array of radiation detectors coupled to a delay line so as to reduce the number of signal conduits traveling through an inner lumen of the catheter body. As subsequently used herein, “array” will be used to mean a plurality of individual detectors, a one-dimensional array of detectors, or a plurality of one-dimensional arrays of detectors. By reducing the number of transmission lines, the profile of the catheter body can be reduced and the inner lumen of the catheter body can be better utilized to deliver medicants, receive a guidewire, or the like. Additionally, the reduction of number of transmission lines allows the catheter body to be reduced in size so as to allow the catheter to access small and tortuous regions not accessible to conventional assemblies.

The delay line can time encode or position encode the signals from each of the individual radiation detectors with respect to a reference or common signal. The encoded signals may then be delivered sequentially down the one delay line. Such “phased” delivery of the signals allow the signals from the array of detectors to be delivered down a smaller number of transmission lines. The signal delivered from the detector will appear at the signal processor at the end of a delay time interval, td with respect to the reference signal. The signal processor can be programmed to know the source of each of the signal based on its time of arrival relative to the reference signal. Delay times can range from a few nanoseconds to a few microseconds, or more.

After the signals have been fed through the delay line, the encoded signals can then be delivered to a signal processor. The pattern of the radiation in the body lumen can thereafter be displayed on an output device.

In exemplary embodiments, the present invention can be used to determine the biological profile of the plaque by delivering beta radiation or gamma radiation-bearing markers into the blood stream with radio markers that bind preferentially to the vulnerable plaque within the body lumen. The precise location and extent of the vulnerable plaque can then be assessed by imaging the gamma ray emissions or beta ray emissions of the radioisotopes using the imaging catheter with the array of radiation detectors.

In another aspect, the array of radiation detectors will include semiconductor radiation detectors. In preferred embodiments, the detectors can include a wafer of cadmium telluride, gallium arsenide, germanium, silicon, mercuric iodide, or the like. The semiconductor wafer is typically disposed between an electron collecting electrode and a hole collecting electrode. When an incident gamma ray or beta ray interacts in the semiconductor detector, a positive and negative charge cloud is generated within the semiconductor material. A voltage applied to the electrodes causes the charged cloud of positive (holes) and negative (electrons) charges to separate and drift to the electrodes. An output signal that is proportional to the amount of energy deposited by the gamma or beta ray by the interacting particles is delivered through the delay line to the signal processor.

In another specific arrangement, the catheters of the present invention use an array of optical detectors. The optical detectors can include a scintillator that is coupled to at least one light detecting diode. When the radioactive emissions interact in the scintillator, the scintillator produces a pulse of electromagnetic radiation that is typically within the visible spectrum. The light detecting diodes will receive the electromagnetic energy and generate an output signal that can be directed into a delay line and subsequently processed by the signal processor. Because the energy of the incoming radiation is converted directly to an electrical signal within the distal portion of the catheter body, the signal(s) are collected without the need of cumbersome light-pipe arrangements. Thus, the optical detectors can yield continuous arrays with a high density of discrete image elements.

In exemplary embodiments, the delay line, which has an inductance and capacitance will delay the travel of the output signals from the radiation detectors (e.g., semiconductor detectors, scintillator and optical detectors, and the like) to the amplifier and signal processor through a connection that is actively or passively impedance matched to the delay line. The delay time between output signals will typically be between approximately 1 nanosecond and 100 nanoseconds. Each individual detector will produce an electric signal that has a delay based on its position within the array. The reference signal delivered from a common electrode, reference electrode, or from a ground plane of the delay line reaches the signal processor prior to the detectors at either end of the chain, or even in the middle of the chain of detectors can be used to provide for position sensitivity along the detectors.

In a further aspect, the present invention provides kits that comprise a catheter having an array of radiation detectors. The radiation detectors can be semiconductor radiation detectors, optical detectors, or the like. The kits can further include instructions for use setting forth any one of the methods described herein. The kits will typically include packaging suitable for containing the catheter and the instructions for use. Exemplary containers include pouches, trays, boxes, tubes, and the like. The instructions for use may be provided on a separate sheet of paper or other medium. Optionally, the instructions may be printed in whole or in part on the packaging. Usually, at least the catheter will be provided in a sterilized condition. Other kit components, such as a guidewire, may also be included.

For a further understanding of the nature and advantages of the invention, reference should be made to the following description taken in conjunction with the accompanying drawings.

### BRIEF DESCRIPTION OF THE DRAWINGS

FIG. 1 illustrates a simplified catheter disposed within a body lumen having radio label markers;

FIG. 2 illustrates an exemplary array of semiconductor radiation detectors;

FIG. 3 illustrates a simplified array of discrete semiconductor radiation detectors;

FIG. 4 illustrates a plurality of one dimensional array of semiconductor radiation detectors;

FIG. 5 illustrates a simplified catheter having an array of optical radiation detectors;

FIG. 6 illustrates a simplified optical detector array having a plurality of discrete optical detectors;

FIG. 7 illustrates a simplified optical detector array having a plurality of one dimensional optical detectors; and

FIG. 8(a) to (c) illustrate various delay line and reference line configurations.

### DESCRIPTION OF THE SPECIFIC EMBODIMENTS

The present invention relates generally to in vivo evaluation of marked lesions in body lumens using a catheter having an array of radiation detectors. More particularly, the present invention provides catheters and methods which detect radiotracers which have bound to vulnerable plaque present in human blood vessels. The radiation detectors can be coupled to a signal processor through a delay line so as to reduce the number of transmission lines traveling through the catheter to the outside of the body.

As will be appreciated by those versed in the art, while the present invention will find particular use in the diagnosis of lesions within blood vessels, the present invention will also be useful in a wide variety of diagnostic and therapeutic procedures. The methodology of plaque detection can be extended to the detection of malignancies following the administration of a metabolic or specific radiolabeled agents (e.g., labeled amino acids, labeled glucose, labeled nucleotides and nucleosides, or the like). Examples of such applications include the differentiation of malignant from benign polyps following virtual colonoscopy and of lung carcinoma from benign anatomy following lung screening by X-ray CT.

The present invention relies generally on introducing a labeled marker, typically a radiolabeled marker with a binding agent, to the patient's blood vessel in such a way that the marker localizes within a lesion or target site which enables assessment of the type of plaque within the blood vessel. Introduction of the labeled marker can be systemic (e.g., oral ingestion, injection or infusion to the patient's blood circulation, and the like), through local delivery (e.g. by catheter delivery directly to a target site within the blood vessel), or a combination of systemic and local delivery.

After introduction of the marker to the patient, the marker can be taken up by the lesion at the target site and the amount of the marker, rate of uptake, distribution of the marker, or other marker characteristics can be analyzed to evaluate the severity of the lesion. The types of radio tracers and radio labels are more fully described in co-pending U.S. patent application No.___________ , filed Sep. 26, 2000, and titled “Methods and Apparatus for Characterizing Lesions in Blood Vessels and Other Body Lumens,” (Attorney Docket No. STAN-158), licensed to the assignee of the present application, the complete disclosure of which is incorporated herein by reference.

Detection and analysis of the label and its pattern within the body lumen will be performed in vivo using an intraluminal catheter. Because the radiation detection is performed in close proximity to the source of radiation, the measurement of radiation intensity can be very accurate.

The catheters according to the present invention will comprise catheter bodies adapted for intraluminal introduction through the body lumen to the target site. The dimensions and other physical characteristics of the catheter bodies will vary significantly depending on the body lumen which is to be accessed. In the exemplary case, the catheter bodies will typically be very flexible and suitable for introduction over a guidewire to a target site within the vasculature. In particular, catheters can be intended for “over-the-wire” introduction when a guidewire lumen extends fully through the catheter body (or separate guidewire lumen) or for “rapid exchange” introduction where the guidewire lumen extends only through a distal portion of the catheter body or the distal tip. In other cases, it may be possible to provide a fixed guidewire at the distal tip of the catheter or even dispense with the guidewire entirely. For convenience of illustration, guidewires will not be shown in all embodiments, but it should be appreciated that they can be incorporated into these embodiments.

Exemplary catheter bodies intended for intravascular introduction will typically have a length in the range from 10 cm to 200 cm and an outer diameter in the range from 1 French (0.33 mm: Fr.) to 24 Fr., usually from 1 Fr. to 20 Fr. In the case of coronary catheters, the length is typically in the range from 80 cm to 150 cm, the diameter is preferably below 8 Fr., more preferably below 6 Fr., and most preferably in the range from 2 Fr. to 4 Fr. Catheter bodies will typically be composed of a polymeric which is fabricated by conventional extrusion techniques. Suitable polymers include polyvinylchloride, polyurethanes, polyesters, poyolefins, polytetrafluoroethylenes (PTFE), silicone rubbers, poyamides, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more lumens being provided when desired. The catheter diameter can be modified by heat expansion and shrinkage using conventional techniques. The resulting catheters will thus be suitable for introduction to the vascular system, often the coronary arteries, by conventional techniques.

The catheters of the present invention will typically have an array of radiation detectors that are capable of detecting ionizing radiation from a radio-isotopic label within a distance between approximately 0.5 cm and 2 cm from the radio label. The radiation detectors can be sized and configured to be able to image a length of the body lumen between approximately 1 cm and 5 cm.

The detector arrays can be assembled from a plurality of individual detectors, a one-dimensional arrays of detectors, or a plurality of one-dimensional array of detectors. The choice of the arrangement of the radiation detectors will depend on the desired bending radius of the distal portion of the catheter body. As can be appreciated, a one-dimensional array of detectors would reduce the bending radius and flexibility of the distal portion catheter body, while a plurality of individual detectors would increase the bending radius and flexibility of the catheter. The electrodes cover substantially all or most of a face of the semiconductor.

In exemplary embodiments, the catheter will include an array of semiconductor radiation detectors or optical detectors. A more complete description of a suitable semiconductor radiation detector is described in U.S. Pat. No. 4,255,659 to Kaufmnan et al., and D. Chu et al. “An Evaluation of Cadmium Telluride Detectors for Computer Assisted Tomography,” Journal of Computer Assisted Tomography, 2:586, (1978), the complete disclosures of which are incorporated herein by reference. A discussion of the timing characteristics of room temperature semiconductors, such as CdTe, can be found in Kaufman, L., Williams S. H., Hosier, K. E., and Ewins, J. H., An Evaluation of Semiconductor Detectors for Positron Cameras (Abstract),J. Comput. Assist. Tomography, Vol. 2, No. 651 (1978) and Kaufmnan L. Williams S. H., Hosier K. E., and Ewins, J. H., An Evaluation of Semiconductor Detectors for Positron Tomography, IEEE Trans. Nucl. Sci. NS-26, No. 648, (1979), the complete disclosures of which are incorporated herein by reference.

In one particular arrangement, the semiconductor detectors includes a semiconductor layer disposed between a continuous electrode and electrically discontinuous electrodes along the axis which spatial resolution is desired. In exemplary embodiments, the continuous electrode is the electron-collecting electrode. The continuous electrode can be used to deliver the biasing voltage for the semiconductor detector and to obtain the reference timing signal. The electrically discontinuous electrodes can be fabricated in a number of ways. The electrodes can be formed through painting, chemical deposition, vapor deposition, dipping, photolithography, etc., of selected portions of the semiconductor. Alternatively, a continuous conductive electrode can be applied to the semiconductor and breaks can be created through scratching, grinding, cutting, or chemically etching. Ion implantation can also be used by implanting either the necessary dopants or insulators as needed. It should be appreciated that other conventional or proprietary methods can be used to create the plurality of electrodes.

In other exemplary embodiments, the radiation detectors include a scintillator coupled to a plurality of optical detectors, such as photodiodes. The scintillator can be positioned at a distal portion of the catheter body to receive energy deposited by an energetic particle. As the particle moves through the scintillator, it emits a visible or ultraviolet light. An array of optically isolated light detectors coupled to the scintillator within the catheter body detect the light produced in the scintillator and generate an electrical signal in response to light.

In conventional catheters, a signal wire is typically needed for each individual radiation detector to couple the detectors to a signal processor. Given the limited space within the inner lumen of the catheter body, the amount of conductive leads disposed within the lumen becomes a limiting factor of the design of the detection catheter. To overcome such a limitation, most embodiments of the present use a delay line to generate a time encoded delivery of the signals from each of the individual detectors to the signal processor. The delay line can time encode the signal from each of the individual detectors with respect to a common or reference electrode. One exemplary embodiment uses an lumped constant (LC) delay line. The delay line can be produced by known microfabrication techniques.

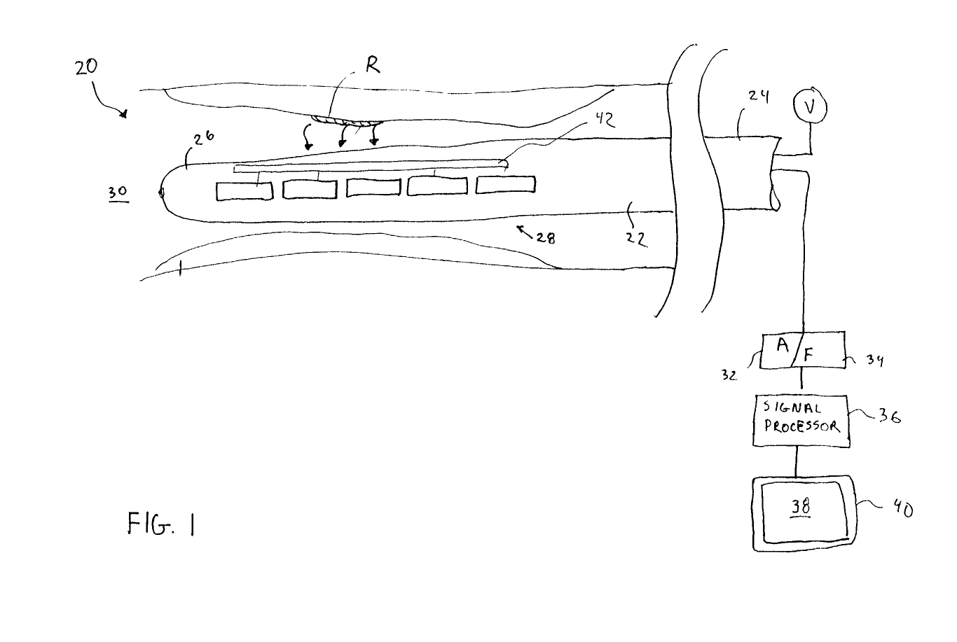

FIG. 1 illustrates an imaging catheter of the present invention. The catheter 20 includes a catheter body 22 having a proximal portion 24 and a distal portion 26. An array of radiation detectors 28 are disposed on the distal portion of the catheter body to detect radioactive markers R within the body lumen 30. The array of radiation detectors 28 can be electrically coupled through a delay line 42 to an amplifier 32 and filter 34, and signal processor 36. The signal processor can process the electrical signals synthesize an image 38 of the radiation pattern in the body lumen on a display output 40. The radiation pattern will typically be a series of peaks separated by valleys.

In the embodiments, the radiation detected by the radiation detectors 28 are converted into electrical signals within the catheter body 22, and the electrical signals are delivered to an amplifier, filter and signal processor that is disposed outside of the patient's body.

In most embodiments, the array of radiation detectors 28 are coupled to a delay line 42 that can time encode the signals received from each of the individual radiation detectors. The delay line 42 can delay the output signal from each of the radiation detectors such that the signals can be delivered to the signal processor through a single transmission line. The source of each of the output signals is determined by the signal processor by its time of arrival relative to a reference signal.

In the exemplary embodiment, the array of detectors 28 are semiconductor radiation detectors. In a particular arrangement, the radiation detectors are room temperature semiconductor CdTe detectors. Timing characteristics of room temperature semiconductors, such as cadmium telluride detectors are more fully described in L. Kaufmnan et al. “An Evaluation of Semiconductor Detectors for Positron Tomography,” IEEE Trans. Nucl. Sci. NS-26:648 (1979), the complete disclosure of which is incorporated herein by reference. Other exemplary semiconductor radiation detectors include gallium arsenide, germanium, silicon, mercuric iodide, or the like.

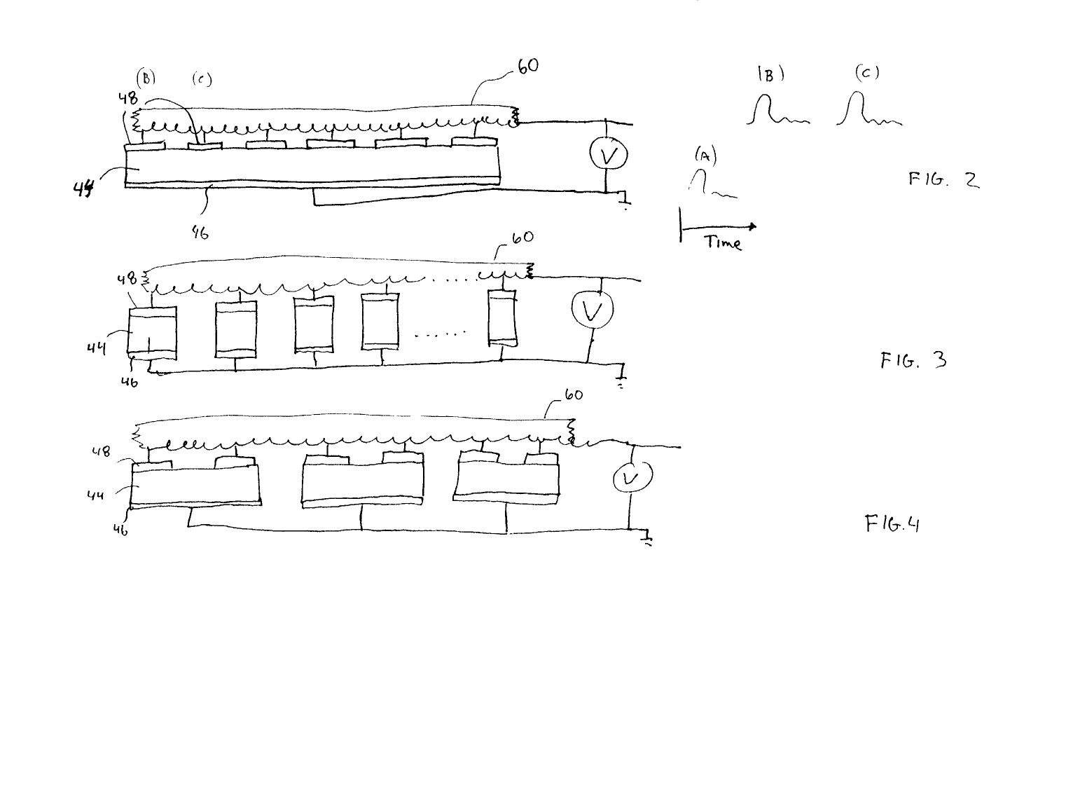

As illustrated in FIG. 2, the semiconductor electrode includes a wafer 44 of the semiconductor material that is disposed between conductive electrodes 46, 48. The conductive electrodes can be formed of platinum, aluminum, copper, gold, or the like. An exemplary one-dimensional array of electrodes includes a continuous electrode 46 that is used as a common, electron collecting electrode for energy measurement and reference timing for the remainder of the individual detectors 48. The individual detectors 48 can be coupled to a lumped-constant delay line 42 comprising of a chain of inductors and capacitors to connect the detectors 28 and signal processor 36.

A high voltage power source can generate a biasing voltage that can be applied through the common electrode 46. In exemplary embodiments, the biasing voltage is between approximately 10 V and 100 V. This voltage is applied through a resistor with a resistance of a few kilomegaohms (>10⁹ ohms). Should a short occur or the device exposed to body fluids, the maximum current flow would be on the order of 30 nanoamps. The applied voltage at the detector side of the resistor would drop to a fraction of a microvolt.

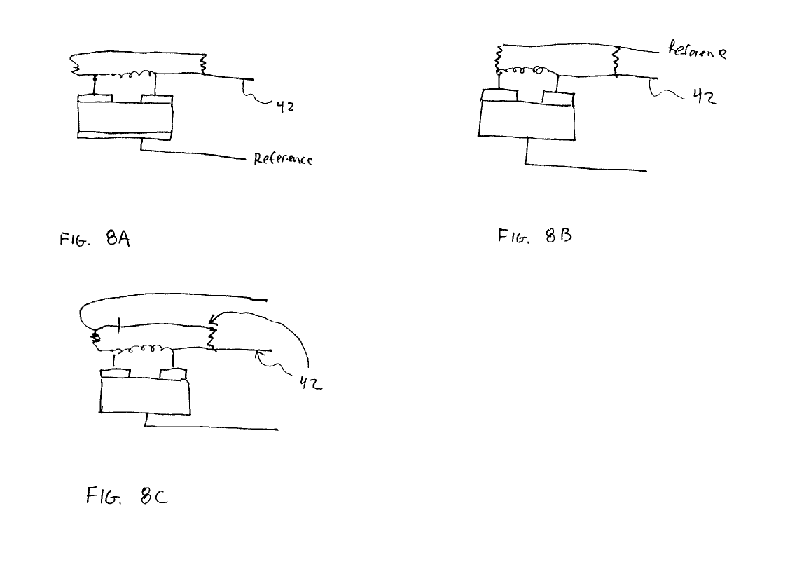

The delay line will typically have a characteristic impedance between approximately 1 kohm and 10 kohm and the output signals of the radiation detectors 28 will typically be brought to the amplifier through a connection that is actively or passively impedance matched to the delay line. The delay time between output signals will typically be between approximately 1 nanosecond and 100 nanoseconds. Each individual detector will produce an electric signal that has a delay based on its position within the array. The reference signal reaches the signal processor prior to the detectors at either end of the chain, or even in the middle of the chain of detectors. The preferred embodiment generates the reference signal from the electron collecting electrode, since that signal has the best characteristics for energy measurement (FIGS. 8a to 8c) and the delay line is connected to the other electrode. Nevertheless, these two can be reversed. Furthermore, the bias voltage is preferentially applied to the continuous electrode, but need not be in all embodiments. Additionally, a reference signal can be obtained from the ground plane 60 of the delay line, or from the other end of the delay line. The latter option has the disadvantage of requiring an additional line, but increases the spatial resolution under some operating circumstances. One suitable delay line is described in L, Kaufmnan et al., “Delay Line Readouts for High Purity Germanium Medical Imaging Cameras,” IEEE Trans. Nucl. Sci. NS-21:652 (1974), the complete disclosure of which is incorporated herein by reference.

In alternative configurations illustrated in FIGS. 3 and 4, the array of radiation detectors can include a plurality of arrays of detectors or a plurality of discrete individual detectors. In any of the embodiments, the detector electrodes 46 can have a spacing between approximately 0.1 mm and 2 mm between adjacent electrodes 48.

The output signals from the semiconductor detectors 28 can be coupled to an amplifier 32 and a filter 34 to deliver a signal indicative of the charge generated within the detector(s). Frequency responses not within a predetermined range of frequencies can be filtered from the output signals. After passing through the amplifier 32 and filter 34, the output signals are delivered to the signal processor 36. The signal processor 36 may be any suitably programmed data processor or computer for reconstructing the imaged body lumen that is coupled to an output display 40.

In one exemplary embodiment, the signal processor 36 uses time to amplitude converters (TACs). The TACs are commercially available in NIM and CAMAC modules. The signals can be shaped in the amplifier 32 and fed into the TAC signal processor. The output is a voltage that is proportional to time. An analog-to-digital converter (ADC) (not shown) can convert the voltage to a digital signal, if needed. The analog signal can be fed into an oscilloscope (not shown) or the like to a real time display, or into a storage scope.

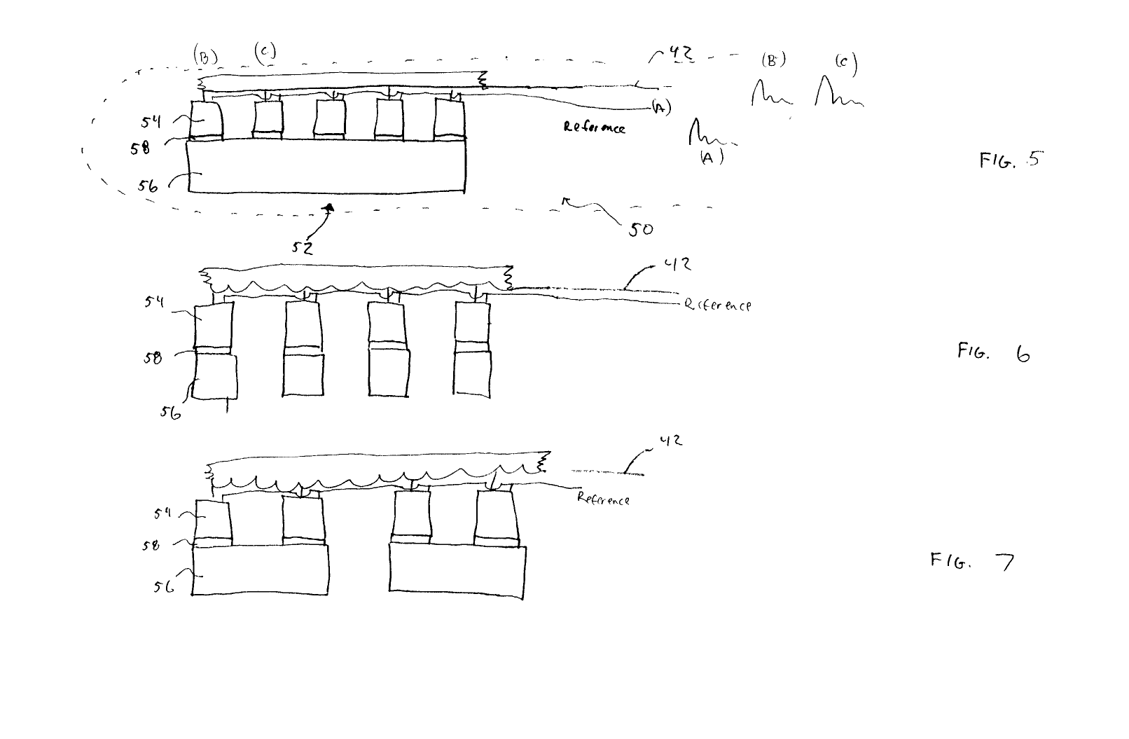

In another aspect, the present invention provides imaging catheters having an array of optical radiation detectors. As shown in FIG. 5, the imaging catheter 50 includes an array of optical detectors 52 coupled to a delay line 42. The optical detectors 52 will typically include a plurality of light detectors 54 (such as a photodiode) that are optically coupled to at least one scintillator 56.

Similar to the previous embodiments, the array of optical detectors can take many forms. For example, the optical detectors can be comprised of a one dimensional array of optical detectors, a plurality of discrete, individual optical detectors (FIG. 6), or a plurality of one dimensional arrays of optical detectors (FIG. 7). The one dimensional array on a single crystal requires less fabrication effort and provides better spatial resolution and sensitivity. On the other hand, the one dimensional array will be more rigid than the plurality of individual detectors.

The scintillator 56 (e.g., doped plastic, optical fiber, crystal, or the like) can be coupled to optically isolated and physically separate photoelectric light detectors 54. These light detectors 54 can be positioned along a desired length of the catheter and coupled to the scintillator either directly or through an index matching material 58. The index matching material can be an optical gel, adhesive, or the like.

The catheter can be advanced to the target site in the body lumen using conventional methods. When a gamma or beta ray associated with the radiation in the body lumen strikes the scintillator, the radiation is absorbed and the scintillator produces a pulse of light. The pulses of light are then transmitted to the photo detectors which convert the pulses of light into electric signals that provide a measurement of the energy of the radiation impinging on the scintillator.

The output signals from each of the individual light detectors 54 can then be fed into the delay line 42 to time encode the output signal. The output signals from the light detectors 28 can then be transmitted down the catheter body and fed into an amplifier and a filter to deliver the signal to the signal processor.

Because the light pulse generated by the impinging radiation diffuses within the scintillator 56, a plurality of the light detectors 54 may be affected by the light pulse generated in the scintillator. Consequently, the delay line and signal processor can be configured to generate a signal that follows a centroid of the light pulse so that the spatial resolution of the one dimensional array can be better than the interconverter distance. For example, for a light pulse occurring between converters, the light will be split in approximately inverse square of the ratio of distances, the closer converter getting more of the light.

While all the above is a complete description of the preferred embodiments of the inventions, various alternatives, modifications, and equivalents may be used. For example, many types of detectors that can be used with the catheters of the present invention. If cost is of major consideration, an inexpensive silicon diode can be used. A silicon diode has better sensitivity in the range of wavelengths of 0.8 to 0.9. μm. One such silicone diode is a silicon PIN diode which has linear responsivity over a wide range of optical powers. Thus, when a detector with linear responsivity is desired, a silicon PIN diode may be preferable. On the other hand, a silicon avalanche diode can detect lower light power than a PIN diode. Therefore, when very low light power is to be detected, a silicon avalanche diode may be preferable.

Although the foregoing invention has been described in detail for purposes of clarity of understanding, it will be obvious that certain modifications may be practiced within the scope of the appended claims.

What is claimed is:

1. A catheter for detecting radioactive markers within a body lumen, the catheter comprising:
a catheter body comprising a proximal portion and a distal portion;
a scintillator positioned at the distal portion of the catheter body that produces light in response to impinging radiation from the radioactive markers within the body lumen; and
an array of optically isolated light detectors optically coupled to the scintillator within the catheter body, wherein the light detectors detect the light produced in the scintillator and generate an electrical signal in response to light.

2. The catheter of claim 1 comprising a delay line electrically coupled to the array of light detectors that time encode the electrical signals from each of the light detectors relative to a reference electrode signal.

3. The catheter of claim 2 further comprising a signal processor coupled to the delay line to process the encoded signal.

4. The catheter of claim 2 wherein the encoded signal of the delay line follows a centroid of the light generated within the scintillator(s).

5. The catheter of claim 2 further comprising an amplifier that amplifies the signal delivered by the delay line.

6. The catheter of claim 2 further comprising a filter that allows only frequency components within a predetermined range of frequencies to contribute to encoded signal delivered by the delay line.

7. The catheter of claim 1 wherein the light detectors comprise a plurality of photodiodes.

8. The catheter of claim 1 wherein the scintillator comprises a crystal, a plastic, a liquid, or an optical fiber.

9. The catheter of claim 1 wherein the scintillator comprises a plurality of scintillators, wherein each of the scintillators is coupled to at least one of the light detectors.

10. The catheter of claim 1 wherein the scintillator is coupled to the light detectors through an index matching material.

11. The catheter of claim 10 wherein the index matching material comprises a gel or an adhesive.

12. The catheter of claim 1 wherein the light detectors are directly coupled to the scintillator(s).

13. A method of detecting a marker within a body lumen, the method comprising:
attaching the radiolabel onto vulnerable plaque in the body lumen;
introducing a catheter having at least one scintillator and a plurality of light detectors into the body lumen; and
generating an electric signal within the light detectors in response to light created by the radiation that interacts with the scintillator.

14. The method of claim 13 further comprising:
time encoding the electric signals from the plurality of light detectors through a delay line, wherein the electric signals from each of the light detectors is time encoded with respect to a reference electrode signal; and
processing the signal received from the delay line.

15. The method of claim 13 wherein transmitting the electric signals is performed in the catheter in vivo.
